(19) United States
(12) Patent Application Publication
(10) Pub. No.: US 2010/0008952 A1
(43) Pub. Date: Jan. 14, 2010

(54) Vaccines for the Rapid Response to Pandemic Avian Influenza

(76) Inventors: Andrea Gambotto, Pittsburgh, PA (US); Paul D. Robbins, Mt. Lebanon, PA (US); Gao Wentao, Pittsburgh, PA (US); Simon Barratt-Boyes, Pittsburgh, PA (US); Adam Soloff, Pittsburgh, PA (US)

Correspondence Address: BAKER BOTTS L.L.P., 30 ROCKEFELLER PLAZA, 44TH FLOOR, NEW YORK, NY, 10112-4498, US

(21) Appl. No.: 12/509,167

(22) Filed: Jul. 24, 2009

Related U.S. Application Data
(63) Continuation of application No. 11/298,102, filed on Dec. 9, 2005.
(60) Provisional application No. 60/634,660, filed on Dec. 9, 2004.

(51) Int. Cl. A61K 39/145; C12N 15/63

(52) U.S. Cl. 424/209.1; 435/320.1

(57) ABSTRACT

The present invention relates to adenovirus-based vaccines against avian influenza viruses with pandemic potential. The present invention provides replication-defective adenoviral vectors, each having a nucleic acid encoding an influenza A polypeptide. When introduced into a subject, the expressed influenza A polypeptide induces the production of antibodies that bind to influenza.

The present invention also provides methods for inducing an immune response in a subject. Subjects are administered a replication-defective adenoviral vector, wherein the vector has a nucleic acid encoding an influenza A polypeptide. When the vector is expressed in the subject, the influenza A polypeptide induces the subject to produce antibodies to influenza.

### CROSS-REFERENCE TO RELATED APPLICATIONS

This application is a continuation of currently pending U.S. patent application Ser. No. 11/298,102, filed Dec. 9, 2005, which claims the benefit of U.S. Provisional Patent Application No. 60/634,660 filed Dec. 9, 2004, both of which are incorporated herewith in its entirety.

### 1. FIELD OF THE INVENTION

The present invention relates to influenza vaccination, and, in particular, to the rapid development of vaccines in response to pandemic avian influenza and a method of inducing an immune response in a subject.

### 2. BACKGROUND OF THE INVENTION

Wild waterfowl, the natural hosts of all known influenza A viruses, are the source of viruses that cause sporadic outbreaks of highly fatal disease in domestic poultry. The recent emergence of highly pathogenic avian influenza (HPAI) strains in poultry and their subsequent transmission to humans in southeast Asia, with frequent outbreaks in poultry leading to the destruction of hundreds of millions of animals, has raised concerns about the potential pandemic spread of lethal disease. Li et al., Nature, 2004, 430:209-213; Yuen et al., Lancet, 1998, 351:467-471. In 1997, highly pathogenic avian influenza H5N1 was transmitted from poultry to humans in Hong Kong, resulting in eighteen infected people and six deaths, and reemerged in 2003 causing two similar cases with one fatality. Yuen, supra; Nicholson et al., Lancet, 2003, 362:1733-1745. In 2003-2005, extensive outbreaks of HPAI H5N1 occurred in nine Asian countries resulting in 19 human cases in Thailand, 91 in Vietnam, seven in Indonesia, and four in Cambodia, with a total of 62 reported deaths. Furthermore, H5N1 infections in family clusters have raised the possibility of human-to-human transmission. As human exposure to and infection with H5N1 viruses continues to increase, so, too, does the likelihood of the generation of an avian-human reassortment virus that may be transmitted efficiently within the global human population, which currently lacks H5N1 specific immunity. Such reassortment events between avian-human and swine-human influenza A viruses have been associated with the 1957 and 1968 influenza pandemics; the 1918 pandemic events remain unclear.

Concern over the potential for the generation of a pandemic H5 strain and its concomitant morbidity and mortality are spurring the search for an effective vaccine. Although conventional inactivated H5 vaccines continue to be evaluated in clinical trials, limited production capability of conventional inactivated influenza vaccines could severely hinder the ability to control the pandemic spread of avian influenza through vaccination. Thus, alternative approaches that provide rapid and effective options against unforeseeable future outbreaks are urgently needed. Current strategies of influenza vaccination are limited by the time required to generate vaccines. The present invention provides methods and compositions for the rapid development of vaccines in response to pandemic avian influenza.

### 2.1. Influenza Virus

Influenza viruses consist of three types, A, B, and C. Influenza A viruses infect a wide variety of birds and mammals, including humans, horses, pigs, ferrets, and chickens. Influenza B and C are present only in humans. Animals infected with Influenza A often act as a reservoir for the influenza virus, by generating pools of genetically and antigenically diverse viruses which are transmitted to the human population. Transmission may occur through close contact between humans and the infected animals, for example, by the handling of livestock. Transmission from human to human may occur through close contact, or through inhalation of droplets produced by coughing or sneezing.

The outer surface of the influenza A virus particle consists of a lipid envelope which contains the glycoproteins hemagglutinin (HA) and neuraminidase (NA). The HA glycoprotein is comprised of two subunits, termed HA1 and HA2. HA contains a sialic acid binding site, which binds to sialic acid found on the outer membrane of epithelial cells of the upper and lower respiratory tract, and is absorbed into the cell via receptor mediated endocytosis. Once inside the cell, the influenza virus particle releases its genome, which enters the nucleus and initiates production of new influenza virus particles. NA is also produced, which cleaves sialic acid from the surface of the cell to prevent recapture of released influenza virus particles. The virus incubates for a short period, roughly five days in a typical case, although the incubation period can vary greatly. Virus is secreted approximately one day prior to the onset of the illness, and typically lasts up to three to five days. Typical symptoms include fever, fatigue, malaise, headache, aches and pains, coughing, and sore throat. Some symptoms may persist for several weeks post infection.

Different strains of influenza virus are characterized primarily by mutations in the HA and NA glycoproteins, and thus HA and NA are used to identify viral subtypes (i.e., H5N1 indicates HA subtype 5 and NA subtype 1). As such, influenza vaccines often target the HA and NA molecules. Conventional influenza virus vaccines often utilize whole inactivated viruses, which possess the appropriate HA and/or NA molecule. Alternatively, recombinant forms of the HA and NA proteins or their subunits have been used as vaccines. However, influenza is an RNA virus and is thus subject to frequent mutation, resulting in constant and permanent changes to the antigenic composition of the virus. The antigenic composition refers to portions of the polypeptide which are recognized by the immune system, such as antibody binding epitopes. Small, minor changes to the antigenic composition are often referred to as antigenic drift. Influenza A viruses are also capable of “swapping” genetic materials from other subtypes in a process called reassortment, resulting in a major change to the antigenic composition referred to as antigenic shift. Because the immune response against the viral particles relies upon the binding of antibodies to the HA and NA glycoproteins, frequent changes to the glycoproteins reduce the effectiveness of the immune response against influenza viruses over time, eventually leading to a lack of immunity. The ability of influenza A to undergo a rapid antigenic shift can often trigger influenza epidemics due to the lack of pre-existing immunity to the new strain.

### 2.2. Influenza Vaccines

Because of the ability of influenza viruses to undergo rapid antigenic drift or antigenic shift, new vaccines are periodically required to combat new strains of influenza. An effective vaccine must include the type of influenza virus that is predicted to be prevalent in the upcoming flu season. If the wrong type of influenza is not included, the vaccine will not provide protection against infection. Production of influenza virus vaccines therefore requires prediction of what influenza viruses will be prevalent, and cannot account for sudden antigenic shift. Accordingly, there is a need in the art for a method to quickly generate and produce influenza virus vaccines.

While many influenza A subtypes are capable of infecting birds, the more recent outbreaks of highly pathogenic viruses have been caused by subtypes H5 and H7. The potential antigenic shifts of the virus, and the resulting lack of immunity in the birds, has lead to rapid spread of the virus among bird populations, including domesticated chicken and fowl. As the standard control measure is the culling of all infected or exposed birds, the rapid spread of avian influenza has resulted in the destruction of millions of birds worldwide. Outbreaks of avian influenza can therefore be devastating to affected poultry farms, and result in tremendous monetary losses. Although rare, human infection by avian influenza also occurs. Due to the potential for rapid antigenic shift and rapid spread of the avian influenza virus, there is great concern that a pandemic caused by an avian influenza virus may occur in the future.

The rapid production and administration of recombinant adenovirus-based vaccines to birds and high-risk individuals in the face of an outbreak may serve to control the pandemic spread of lethal avian influenza. The lengthy development time and limited production capability of conventional inactivated influenza vaccines could severely hinder the ability to control the pandemic spread of avian influenza through vaccination. Thus, there is a need in the art for a method of quickly developing and mass producing large quantities influenza vaccine.

The present invention provides for the rapid development of an adenoviral-based influenza A vaccine directed against the hemagglutinin (HA) protein of the A/Vietnam/1203/2004 (H5N1) (VN/1203/04) strain isolated during the 2003-2005 lethal human outbreak in Vietnam. Vaccination of mice induced HA-specific antibodies and broad cellular immunity likely to provide heterotypic immunity. Mice vaccinated with full-length HA were fully protected from a lethal intranasal challenge with VN/1203/04. Moreover, a single subcutaneous immunization completely protected chickens from a massive intranasal challenge with VN/1203/04 capable of killing all control-vaccinated chickens within 2 days.

### 3. SUMMARY OF THE INVENTION

The present invention relates to adenovirus-based vaccines, e.g., an adenoviral-based H5N1 influenza vaccine, against avian influenza viruses with pandemic potential. It is based, at least in part, on studies in mice and chickens which demonstrate that the adenoviral-based vaccine of the invention induce an immune response. The present invention provides replication-defective adenoviral vectors, each having a nucleic acid encoding an influenza A polypeptide. The present invention provides for E1/E3-deleted adenovirus serotype 5-based vectors that express codon-optimized hemagglutinin (HA) gene from A/Vietnam/1203/2004 influenza virus (VN/1203/04). These vectors, according to the invention, may be administered to a subject to induce an immune response, including but not limited to, the production of antibodies that bind to influenza.

The present invention also provides methods for inducing an immune response in a subject. For example, a method according to the invention comprises administering to the subject a replication-defective adenoviral vector, wherein the vector has a nucleic acid encoding an influenza A polypeptide and the expressed influenza A polypeptide induces production of antibodies to influenza in the subject.

### 3.1. DEFINITIONS

As used herein, “avian influenza virus” refers to any influenza virus that may infect birds. “Highly pathogenic avian influenza virus (HPAI)” refers to an avian influenza virus which is highly virulent and characterized by high mortality. In one embodiment, the avian influenza virus is of the H5 subtype. In another embodiment, the avian influenza virus is of the H7 subtype. In another embodiment, the avian influenza virus is of the H5N1 subtype. In one embodiment, the avian influenza virus is A/Vietnam/1203/2004 (H5N1). In another embodiment, the avian influenza virus is A/Hong Kong/156/1996 (H5N1).

As used herein, the term “cDNA” can refer to a single-stranded or double-stranded DNA molecule. For a single-stranded cDNA molecule, the DNA strand is complementary to the messenger RNA (“mRNA”) transcribed from a gene. For a double-stranded cDNA molecule, one DNA strand is complementary to the mRNA and the other is complementary to the first DNA strand.

As used herein, a “coding sequence” or a “nucleotide sequence encoding” a particular protein is a nucleic acid molecule which is transcribed and translated into a polypeptide in vivo or in vitro when placed under the control of appropriate regulatory sequences. The boundaries of the coding sequence are determined by a start codon at the 5′-terminus and a translation stop codon at the 3′-terminus. A coding sequence can include, but is not limited to, prokaryotic nucleic acid molecules, cDNA from eukaryotic mRNA, genomic DNA from eukaryotic (e.g. mammalian) sources, viral RNA or DNA, and even synthetic nucleotide molecules. A transcription termination sequence will usually be located 3′ to the coding sequence.

As used herein, the term “control sequences” refers collectively to promoter sequences, polyadenylation signals, transcription termination sequences, upstream regulatory domains, enhancers and the like, and untranslated regions (UTRs) including 5′-UTRs and 3′-UTRs, which collectively provide for the transcription and translation of a coding sequence in a host cell. As used herein, a control sequence “directs the transcription” of a coding sequence in a cell when RNA polymerase will bind the promoter sequence and transcribe the coding sequence into mRNA, which is then translated into the polypeptide encoded by the coding sequence.

As used herein, the term “gene” refers to a DNA molecule that either directly or indirectly encodes a nucleic acid or protein product that has a defined biological activity.

As used herein, the term “genomic DNA” refers to a DNA molecule from which an RNA molecule is transcribed. The RNA molecule is most often a messenger RNA (mRNA) molecule, which is ultimately translated into a protein that has a defined biological activity, but alternatively may be a transfer RNA (tRNA) or a ribosomal RNA (rRNA) molecule, which are mediators of the process of protein synthesis.

As used herein, two nucleic acid molecules are “functionally equivalent” when they share two or more quantifiable biological functions. For example, nucleic acid molecules of different primary sequence may encode identical polypeptides; such molecules, while distinct, are functionally equivalent. In this example, these molecules will also share a high degree of sequence homology. Similarly, nucleic acid molecules of different primary sequence may share activity as a promoter of RNA transcription, wherein said RNA transcription occurs in a specific subpopulation of cells, and responds to a unique group of regulatory substances; such nucleic acid molecules are also functionally equivalent.

As used herein, a “heterologous” region of a DNA construct is an identifiable segment of DNA within or attached to another DNA molecule that is not found in association with the other molecule in nature. An example of a heterologous coding sequence is a construct where the coding sequence itself is not found in nature (e.g. synthetic sequences having codons different from the native gene). Allelic variation or naturally occurring mutational events do not give rise to a heterologous region of DNA as used herein.

As used herein, two nucleic acid molecules are “homologous” when at least about 60% to 75% or preferably at least about 80% or most preferably at least about 90% of the nucleotides comprising the nucleic acid molecule are identical over a defined length of the molecule, as determined using standard sequence analysis software such as Vector NTI, GCG, or BLAST. DNA sequences that are homologous can be identified by hybridization under stringent conditions, as defined for the particular system. Defining appropriate hybridization conditions is within the skill of the art. See e.g. Current Protocols in Molecular Biology, Volume I, Ausubel et al., eds. John Wiley: New York N.Y., first published in 1989 but with annual updating, wherein maximum hybridization specificity for DNA samples immobilized on nitrocellulose filters may be achieved through the use of repeated washings in a solution comprising 0.1-2×SSC (15-30 mM NaCl, 1.5-3 mM sodium citrate, pH 7.0) and 0.1% SDS (sodium dodecylsulfate) at temperatures of 65-68° C. or greater. See also Sambrook et al. Molecular Cloning: A Laboratory Manual, Cold Spring Harbor Laboratory, 1989. For DNA samples immobilized on nylon filters, a stringent hybridization washing solution may be comprised of 40 mM NaPO₄, pH 7.2, 1-2% SDS and 1 mM EDTA. Again, a washing temperature of at least 65-68° C. is recommended, but the optimal temperature required for a truly stringent wash will depend on the length of the nucleic acid probe, its GC content, the concentration of monovalent cations and the percentage of formamide, if any, that was contained in the hybridization solution (Ausubel et al., supra).

As used herein, the term “nucleic acid molecule” includes both DNA and RNA and, unless otherwise specified, includes both double-stranded and single-stranded nucleic acids. Also included are molecules comprising both DNA and RNA, either DNA/RNA heteroduplexes, also known as DNA/RNA hybrids, or chimeric molecules containing both DNA and RNA in the same strand. Nucleic acid molecules of the invention may contain modified bases. The present invention provides for nucleic acid molecules in both the “sense” orientation (i.e. in the same orientation as the coding strand of the gene) and in the “antisense” orientation (i.e. in an orientation complementary to the coding strand of the gene).

As used herein, the term “operably linked” refers to an arrangement of nucleic acid molecules wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

As used herein, the term “sequence” refers to a nucleic acid molecule having a particular arrangement of nucleotides, or a particular function, e.g. a termination sequence.

As used herein, exogenous DNA may be introduced into a cell by processes referred to as “transduction,” “transfection,” or “transformation.” Transduction refers to the introduction of genetic material, either RNA or DNA, across the membrane of a eukaryotic cell via a vector derived from a virus. Transfection refers to the introduction of genetic material across the membrane of a eukaryotic cell by chemical means such as by calcium phosphate-mediated precipitation, by mechanical means such as electroporation, or by physical means such as bioballistic delivery. Transformation refers to the introduction of genetic material into non-eukaryotic cells, such as bacterial cells or yeast cells, by chemical, mechanical, physical or biological means. The genetic material delivered into the cell may or may not be integrated (covalently linked) into chromosomal DNA. For example, the genetic material may be maintained on an episomal element, such as a plasmid. A stably transformed non-eukaryotic cell or stably transfected eukaryotic cell is generally one in which the exogenous DNA has become integrated into the chromosome so that it is inherited by daughter cells through chromosome replication, or one which includes stably-maintained extrachromosomal plasmids. This stability is demonstrated by the ability of the cell to establish clones comprised of a population of daughter cells containing the exogenous DNA. Cells containing exogenous DNA that is not integrated into the chromosome or maintained extrachromosomally through successive generations of progeny cells are said to be “transiently transformed” or “transiently transfected.”

As used herein, the term “subject” or “patient” refers to an animal, e.g., a bird or mammal. In one embodiment, the subject is a human. In another embodiment, the subject is a domesticated bird, such as a chicken or a duck.

As used herein, the term “derived” means “obtained from,” “descending from,” or “produced by.” In the context of nucleic acids or polypeptides derived from a particular parent source, the term derived refers to the use of the parent source as a template for the nucleic acid sequence or the amino acid sequence. The nucleic acid or polypeptide derived from the parent source may possess all or part of the nucleic acid or amino acid sequence of the parent source, in the presence or absence of deletions, substitutions, or modification.

A “vaccine,” as that term is used herein, is a composition which elicits an immune response (cellular and/or humoral) in a subject. A vaccine may reduce the risk of infection but does not necessarily prevent infection. In specific, non-limiting embodiments, a vaccine increases the level of cellular and/or humoral immunity by at least 30 percent, 50 percent, or 100 percent of baseline levels.

Examples of categories of vaccine include live virus vaccines, where the virus has been weakened, or attenuated, such that it cannot cause disease; killed-virus vaccines; vaccines which contain one or more viral proteins; chimeric viruses whereby a non-pathogenic virus is engineered to contain genetic information encoding immunogenic peptide(s) from a disease-causing virus; and naked DNA encoding such peptides. Of the last two categories of vaccine, the non-pathogenic virus can “deliver” the immunogenic peptides by infecting host cells, and the naked DNA can be injected, for example intramuscularly, into host cells where it can be taken up and ultimately expressed as antigenic protein. The requirements for a vaccine to be effective vary from virus to virus, and depend upon, among other things, whether, and to what degree, humoral and/or cellular immunity is necessary to reduce the likelihood of infection, the genetic variability in the immunogenic regions of a virus, and virulence. Yet another category of vaccines uses self-replicating and self-limiting RNA (“RNA replicons”), which cause lysis of transfected cells and do not raise the concerns associated with naked DNA vaccines, which can integrate into host chromosomes (Cheng et al., 2001, J. Virol. 75(5):2368-2376).

### 4. BRIEF DESCRIPTION OF THE FIGURES

FIG. 1. Immunization with Ad5-based HAs vaccine induces broad virus-specific immune responses and protection in mice. IFN-γ ELISPOT analysis of freshly thawed splenocytes stimulated with overlapping 15-mer peptides consisting of the complete VN1203HA protein and additional non-conserved HK156HA sequences. Shown are responses from two animals per group. A) Total additive cellular immune responses directed against HA1 (black) or HA2 (white) sequences of both VN1203HA and HK156HA. B-D) Distribution of strain specific cellular immunity against pools of peptides comprising the reference VN1203HA strain (VN.A, VN.B, VN.C) or the non-conserved HK156HA sequences (HK.D) for HA1 (black) and HA2 (white) regions. E-G) Characterization of both conserved and strain specific vaccine induced peptide epitopes.

FIG. 2. Humoral immune responses in vaccinated mice. (a) Anti-H5N1 HA IgG antibody responses. Sera from 8 mice per group were collected 8 weeks after the second immunization and tested by ELISA for the presence of H5N1 subtype specific IgG antibodies using purified VN1203HA recombinant protein. Antibody titers are expressed as log 10 value of reciprocal endpoint titers. (b) Serum HI antibody responses. Sera were collected eight weeks after the second vaccination and tested individually for HI antibody against VN/1203/04 (top) or HK/156/97 (bottom) virus. HI antibody titers for individual mice are expressed as a log 2 value of the reciprocal of the highest dilution of serum inhibiting agglutination of 1% horse erythrocytes by 4 HA units of virus. Horizontal lines represent the geometric mean of each group. (c) Kinetics of serum HI antibody production. Ad.VNHA (▴), Ad.VNHA1 (▪), Ad.HKHA1 (X), or empty vector Ad.Ψ5 (⋄).

FIG. 3. Cellular immune responses in vaccinated mice. (a) HA1- and HA2-specific responses of splenocytes taken 3-5 days after a second boost as determined by IFN- [symbol] ELISPOT using pools of 15-mer peptides. (b) Identification of individual epitope specific-responses as determined by IFN-ELISPOT using individual 15-mer peptides as shown. Data represent mean±SEM of triplicate determinations in a minimum of two mice per group. SFC=spot-forming cells.

FIG. 4. Outcome in vaccinated mice following lethal intranasal challenge with VN/1203/04. (a) Weight loss and (b) survival in mice challenged by intranasal inoculation with 100 LD₅₀ of VN/1203/04 influenza virus 8 weeks after the second immunization. Mean weight loss is expressed as a percent of original weight. Ad.VNHA (▴), Ad.VNHA1 (▪), Ad.HKHA1 (X), Ad.VNHA2 (), or empty vector Ad.ψ5 (⋄). (c) Virus titer in different tissues determined 3 and 6 days after challenge for Exp. 1 and Exp. 2, respectively.

### 5. DETAILED DESCRIPTION OF THE INVENTION

The present invention relates to adenovirus-based vaccination against avian influenza viruses. The present invention is based, in part, on the development in 5 weeks of an adenoviral-based influenza vaccine based on the A/Vietnam/1203/2004 (H5N1) strain isolated during the 2003-2004 lethal human outbreak. Vaccinated mice had broad virus-specific immunity and were fully protected from a lethal intranasal H5N1 challenge, whereas all control animals which did not receive the vaccine died within 9 days. Thus, the present invention provides a viable system for rapid production of influenza vaccine utilizing an adenovirus-based vaccination strategy against an avian influenza virus with pandemic potential.

In a nonlimiting embodiment, the present invention provides a replication-defective adenoviral vector comprising a nucleic acid encoding an influenza A polypeptide, wherein the expressed polypeptide, when introduced into a subject, induces the production of antibodies that bind to influenza. In a nonlimiting embodiment, the present invention provides a vector of the invention and a pharmaceutically acceptable carrier. In non-limiting embodiments, the influenza A polypeptide comprises Hemagglutinin (HA) or HA1 subunit or portions thereof. In specific non-limiting embodiments, the influenza A polypeptide comprises any one of the influenza A polypeptides described in the Examples below. In one embodiment, the influenza A polypeptide is derived from A/Vietnam/1203/2004 (H5N1). In another embodiment, the influenza A polypeptide is derived from A/Hong Kong/156/1996 (H5N1).

In another nonlimiting embodiment, the present invention provides a method for inducing an immune response in a subject, the method comprising administering to the subject a replication-defective adenoviral vector, wherein the vector comprises a nucleic acid encoding an influenza A polypeptide, and wherein the polypeptide induces the subject to produce antibodies that bind to influenza. The subject may be an animal (e.g., bird, such as a chicken, duck, turkey, goose, or any other domestic or wild bird, or mammal), preferably a human. Administration may be by any method known in the art. In particular, nonlimiting embodiments, the vector of the invention is administered to the subject intramuscularly, intranasally, or subcutaneously.

The vector and vaccines of the invention may protect high-risk human populations such as healthcare workers and animal handlers. Moreover, given that human adenoviral vectors can induce immunity in chickens, susceptible poultry may be vaccinated in accordance with the methods of the invention. Widespread vaccination can be monitored because of the simultaneous immunity to adenovirus, for example.

The adenoviral-based vaccine of the invention can confer cross-protection to several influenza virus subtypes.

### 5.1. Adenoviral Vectors

The present invention also relates to replication-defective adenoviral vectors, for use in delivering nucleic acids encoding an influenza A polypeptide operably linked to expression control sequences such that the influenza A polypeptide can be expressed.

Adenoviruses are non-enveloped DNA viruses, which are stable, easy to manipulate, and are easily grown at high titers. Deletion of genes from the adenoviral genome also allow for the insertion of large pieces of foreign DNA. These traits make adenoviruses very desirable as vectors for delivery of foreign DNA into a host cell. The terms “adenovirus vector” and “adenoviral vector” are used interchangeably in this specification, and refer to a polynucleotide construct of the present invention. A polynucleotide construct of this invention may be in any of several forms, including, but not limited to, DNA, DNA encapsulated in an adenovirus coat, DNA packaged in another viral or viral-like form (such as herpes simplex, and AAV), DNA encapsulated in liposomes, DNA complexed with polylysine, complexed with synthetic polycationic molecules, conjugated with transferrin, and complexed with compounds such as PEG to immunologically “mask” the molecule and/or increase half-life, and conjugated to a nonviral protein. As used herein, the term “DNA” includes the standard bases A, T, C, and G, as well as any analogs or modified forms of these bases, such as methylated nucleotides, internucleotide modifications such as uncharged linkages and thioates, use of sugar analogs, and modified and/or alternative backbone structures, such as polyamides.

The adenoviral vector is deficient in at least one gene function that is required for viral propagation (i.e., an essential adenoviral gene function), rendering it replication-deficient. The replication-deficient adenoviral vector may be incubated in a cell in which complements the defective gene function to allow propagation of the replication-deficient adenoviral vector when. The adenoviral vector may be deficient in at least one essential gene function of the E1 region of the adenoviral genome that is required for viral replication. The adenoviral vector may be deficient in one or more essential gene functions in two or more regions of the adenoviral genome. For example, the adenoviral vector may be deficient in one or more of the E1, E2, E3, or E4 regions. In one embodiment, the adenoviral vectors are deficient in the E1 and E3 regions.

Sources for the adenoviral vector DNA include any species, strain, subtype, or mixture of species, strains, or subtypes, of an adenovirus or a chimeric adenovirus. The adenoviral vector can be any adenoviral vector capable of growth in a cell, which is in some significant part (although not necessarily substantially) derived from or based upon the genome of an adenovirus. The adenoviral vector preferably comprises an adenoviral genome of serotype 5.

Nucleic acids may be inserted into the adenoviral vector such that, when a host cell is infected by the adenoviral vector, the polypeptides encoded by the nucleic acids will be expressed. The nucleic acids may include control sequences operably linked to a coding sequence which encodes for a polypeptide. In one embodiment, the coding sequence encodes an influenza polypeptide. In preferred embodiments, the coding sequence encodes polypeptides derived from the A/Vietnam/1203/2004 (H5N1) strain or the A/Hong Kong/156/1996 (H5N1) strain.

The construction of adenoviral vectors and insertion of nucleic acids into the adenoviral vectors is well understood in the art and involves the use of standard molecular biological techniques, such as those described in, for example, Sambrook et al. Molecular Cloning: A Laboratory Manual, Cold Spring Harbor Laboratory, 1989, and Ausubel et al., and other references mentioned herein. Moreover, adenoviral vectors can be constructed and/or purified using the methods set forth, for example, in U.S. Pat. No. 5,965,358 and International Patent Applications WO 98/56937, WO 99/15686, and WO 99/54441.

### 5.2. Influenza Nucleic Acids and Polypeptides

The present invention relates to compositions and/or methods which comprise and/or utilize, respectively, the various nucleic acid molecules that may be derived from influenza viruses. In a preferred embodiment, the influenza virus is an avian influenza virus. In other preferred embodiments, the virus is the A/Vietnam/1203/2004 (H5N1) (hereinafter “VN/1203/04”) strain or the A/Hong Kong/156/1996 (H5N1) (hereinafter “HK/156/97) strain. The nucleic acid may encode the full length or the HA1 or HA2 subunits of the virus.

The HA of influenza A virus is comprises two structurally regions, a globular head region and a stem region. The globular head region contains a sialic acid binding site which is responsible for virus attachment to a target cell and plays a role in the hemagglutination activity of HA. The stem region contains a fusion peptide which allows for membrane fusion between the viral envelope and the outer membrane of the target cell. HA of influenza A virus is activated when the HA is cleaved at one site with a protease, allowing for infection to occur. The larger polypeptide thus obtained is called HA1 while the smaller one HA2.

The nucleic acid may be codon-optimized. Codon optimization a process by which nucleic acid variants of the gene of interest contain codons which have been altered for optimal expression in a given host cell. Particular codon alterations will depend upon the host cell being used. Codon optimization may be performed using readily available software or algorithms, such as the UpGene algorithm (www.vectorcore.pitt.edu/upgene.html). Gao, W. et al. Biotechnol. Prog., 2004, 20:443-448.

The present invention relates to isolated nucleic acids encoding an influenza polypeptide. A gene encoding an influenza viral protein, whether viral genomic DNA or cDNA, can be isolated from any subtype of influenza virus. Methods for obtaining an influenza viral hemagglutinin gene, for example, are well known in the art, as described above (see, e.g., Sambrook et al., supra). Accordingly, any influenza virus subtype potentially can serve as the nucleic acid source for the molecular cloning of an influenza viral gene. The DNA may be obtained by standard procedures known in the art from cloned DNA (e.g., a DNA “library”) by chemical synthesis, by cDNA cloning, or by the cloning of genomic influenza viral DNA, or fragments thereof, purified from the desired cell. (See, for example, Sambrook et al., supra). In preferred embodiments, the genomic influenza viral DNA is obtained from the A/Vietnam/1203/2004 (H5N1) strain or the A/Hong Kong/156/1996 (H5N1) strain.

Once the genomic influenza viral DNA is obtained, DNA fragments may be generated, some of which will encode the desired gene. The DNA may be cleaved at specific sites using various restriction enzymes which are well known in the art. Alternatively, the DNA may be fragmented by use of a DNAse or by physical shearing, for example, by sonication. The linear DNA fragments can then be separated according to size by standard techniques, including but not limited to, agarose and polyacrylamide gel electrophoresis and column chromatography.

Numerous methods well known in the art may be used to identify specific DNA fragments. For example, probes may be used to screen for known sequences via nucleic acid hybridization. For example, oligonucleotides corresponding to the partial amino acid sequence information obtained for the influenza viral protein can be prepared and used as probes for DNA encoding the influenza viral gene, or as primers for cDNA or mRNA (e.g., in combination with a poly-T primer for RT-PCR). Preferably, fragments which are unique to the target influenza viral gene are used as probes. Those DNA fragments with substantial homology to the probe will hybridize. The greater the degree of homology, the more stringent hybridization conditions can be used.

The presence of the gene may be detected by assays based on the physical, chemical, or immunological properties of its expressed product. For example, nucleic acids which can produce proteins with particular antigenic properties may be screened, for example, by measuring binding to antibodies, or by measuring their ability to elicit an immune response.

Influenza viral DNA of the invention can also be identified by hybridization to complementary mRNAs. Such nucleic acid fragments may represent available, purified influenza viral DNA, or may be synthetic oligonucleotides designed from the partial amino acid sequence information. The influenza viral DNA may also be identified by immunoprecipitation analysis or functional assays (e.g., tyrosine phosphatase activity) of the in vitro translation products.

The present invention relates to influenza polypeptides encoded by isolated nucleic acids. This includes a full length protein, or naturally occurring form of an influenza viral protein, and any fragments thereof from any influenza viral source. It is within the abilities of a person of ordinary skill in the art using conventional methods that are well known in the art to select influenza viral proteins, or fragments thereof, based upon their desired properties, such as antigenicity. Non-limiting examples include screening the influenza viral proteins or fragments thereof by screening for their ability to bind to influenza-specific antibodies (e.g. by ELISA), or for their ability to elicit cell-mediated immune responses (e.g., by ELISPOT). In one embodiment the influenza polypeptide is hemagglutinin or subunits thereof. In another embodiment, the influenza polypeptide is HA1.

The production and use of derivatives and analogs related to influenza viral gene products are within the scope of the present invention. Influenza viral gene product derivatives can be made by altering encoding nucleic acid sequences by substitutions, additions or deletions that provide for functionally equivalent molecules. Preferably, derivatives are made that have enhanced or increased antigenic activity relative to native influenza viral protein.

### 5.3. Vaccines

The replication-defective adenoviral vector of the present invention may be used as a vaccine to reduce the risk of infection by influenza. When they are used as vaccines, the replication-defective adenoviral vectors of the present invention are administered to an individual using known methods. Administration can occur using conventional routes of administration and/or by routes which mimic the route by which infection by the pathogen of interest occurs. They can be administered in a vaccine composition which includes, in addition to the replication-deficient adenoviral vector, a physiologically acceptable carrier. The composition may also include an immunostimulating agent or adjuvant, flavoring agent, or stabilizer.

Conventional and pharmaceutically acceptable routes of administration include intranasal, intramuscular, intratracheal, intratumoral, subcutaneous, intradermal, intravenous, rectal, nasal, oral and other parenteral routes of administration. Routes of administration may be combined, if desired, or adjusted depending upon the antigenic peptide or the disease. The vaccine composition can be administered in a single dose or in multiple doses, and may encompass administration of booster doses, to elicit and/or maintain immunity.

The replication-defective adenoviral vector vaccine is administered in an “effective amount,” that is, an amount of replication-defective adenoviral vector that is effective in a selected route of administration to elicit an immune response effective to facilitate protection of the host against infection, or symptoms associated with infection, by a pathogenic organism, i.e., influenza virus. In some embodiments, an “effective amount” of a replication-defective adenoviral vector vaccine is an amount of replication-defective adenoviral vector that is effective in a route of administration to elicit an immune response effective to reduce or inhibit the symptoms associated with influenza virus infection, or to reduce the likelihood that an influenza virus infection will occur.

The amount of replication-defective adenoviral vector in each vaccine dose is selected as an amount which induces an immunoprotective or other immunotherapeutic response without significant, adverse side effects generally associated with typical vaccines. Such amount will vary depending upon the nucleic acid encoded by the vector, whether or not the vaccine formulation comprises an adjuvant, and a variety of host-dependent factors. An effective dose of replication-defective adenoviral vector vaccine will generally involve administration of from about 2×10¹⁰ to about 10×10¹⁰ viral particles. In one embodiment, about 4×10¹⁰ to about 7×10¹⁰ viral particles are administered. In another embodiment, about 5×10¹⁰ viral particles are administered. An optimal amount for a particular vaccine can be ascertained by standard studies involving observation of antibody titers and other responses in subjects. The levels of immunity provided by the vaccine can be monitored to determine the need, if any, for boosters. Following an assessment of antibody titers in the serum, optional booster immunizations may be desired. The immune response to the protein of this invention is enhanced by the use of adjuvant and or an immunostimulant.

### 5.4. Vaccine Compositions

The present invention further provides compositions, including pharmaceutical compositions, comprising the replication-defective adenoviral vector of the invention.

Compositions comprising replication-defective adenoviral vector of the invention may include a buffer. Many suitable buffers are well known in the art, and are a person of ordinary skill in the art will is capable of selecting an appropriate buffer. In some instances, the composition can comprise a pharmaceutically acceptable excipient, a variety of which are known in the art and need not be discussed in detail herein.

When used as a vaccine, a replication-defective adenoviral vector of the invention can be formulated in a variety of ways. In general, the vaccine of the invention is formulated according to methods well known in the art using suitable pharmaceutical carrier(s) and/or vehicle(s). A suitable vehicle is sterile saline. Other aqueous and non-aqueous isotonic sterile injection solutions and aqueous and non-aqueous sterile suspensions known to be pharmaceutically acceptable carriers and well known to those of skill in the art may be employed for this purpose.

Optionally, a vaccine composition of the invention may be formulated to contain other components, including, e.g., adjuvants, stabilizers, pH adjusters, preservatives and the like. Such components are well known to those of skill in the art.

The vaccine compositions of the present invention may contain multiple replication defective adenoviral vectors, each carrying a different influenza virus polypeptide.

### 5.6. Methods of Treating

The present invention also relates to a method for inducing an immune response in a mammal, the method comprising administering to the mammal a replication-defective adenoviral vector, wherein the vector comprises a nucleic acid encoding an influenza A polypeptide, and wherein the polypeptide induces the mammal to produce antibodies that bind to influenza.

The present invention provides methods for eliciting an immune response to an antigen, comprising administering to a subject the replication-defective adenoviral vector carrying a nucleic acid encoding an influenza A polypeptide of the present invention, wherein the replication-defective adenoviral vector enters a cell, the influenza A polypeptide is expressed, and an immune response is elicited to the influenza A polypeptide. The polypeptide may be of variable length, and may be subject to normal host cell modifications such as glycosylation, myristylation, or phosphorylation. The polypeptides may be modified to undergo intracellular, extracellular, or cell-surface expression, for example, by use of a signal sequence.

The replication defective adenovirus vector of the present invention can be administered alone or in the compositions discussed above. The replication defective adenovirus vector of the present invention may be co-administered with other drugs or substance, which may promote DNA uptake or facilitate an immune response.

Using the methods and compositions described herein in connection with the subject invention, an immunoprotective response against an influenza infection may be induced in any subject, human or non-human, susceptible to infection by influenza. Whether an immune response is effective can be determined by standard assays, including, but not limited to, monitoring the progression of influenza symptoms, measuring for influenza specific antibodies, or measuring cells which are secreting influenza antibodies.

Administration of the replication defective adenovirus vector may be performed through any method known in the art, including but no limited to, intravenous, intraperitonial, intradermal, subcutaneous, intramuscular, intranasal, or inhalation. In one embodiment, subjects the replication defective adenovirus vector is administered via the mucosal route by nasal inhalation. Mucosal administration may also be performed by use of nose-drops. Mucosal routes of administration include the nares, trachea, tongue, or mucous membranes.

Once vaccinated, subjects may be monitored to determine the efficacy of the vaccination treatment. Monitoring the efficacy of vaccination treatment may be performed by any method known to a person of ordinary skill in the art. In one embodiment, a blood or fluid sample may be assayed to detect the levels of antibodies directed to influenza. In another embodiment, ELISPOT may be performed to detect an immune response to influenza.

In one embodiment, immunization is achieved with the use of a replication deficient adenovirus vector carrying a nucleic acid encoding for the influenza polypeptide HA or a fragment thereof. In a particular embodiment, the polypeptide is the HA1 subunit from the A/Vietnam/1203/2004 (H5N1) (hereinafter “VN/1203/04”) strain. In another embodiment, the polypeptide is the HA1 subunit from the A/Hong Kong/156/1997 (H5N1) strain.

### 6. EXAMPLES

### 6.1. Example 1

### Generation of Adenoviral Vectors Expressing Influenza

Three E1/E3-deleted adenovirus serotype 5-based vectors were generated. These vectors express codon-optimized influenza A/Vietnam/1203/2004 (H5N1) (VN/1203/04) full length Hemagglutinin (HA) or HA1 sub-unit (Ad.VN1203.HA, Ad.VN1203HA1, respectively) and influenza A/Hong Kong/156/1997 (H5N1) (HK/156/97) HA1 (Ad.HK156HA1). Codon optimization and gene synthesis techniques (Gao, supra) yielded increased expression levels of viral antigens when compared with the wild type sequence and allowed generation of the recombinant transgene without the use of H5N1 virus. Generation of the recombinant adenoviral vectors was completed 36 days after receiving the 2004 Vietnam strain influenza VN/1203/04 HA sequence from the Centers for Disease Control, illustrating the rapidity for adenoviral-based vaccine development in accordance with the present invention.

### 6.1.1. ELISPOT Assay for IFN-γ

Ninety-six well membrane-coated plates (Millipore, Bedford, Mass., USA) were incubated with 10 μg/ml mAb to mouse IFN-γ (AN-18; Mabtech AB, Mariemont, Ohio, USA) in 0.1 M carbonate buffer overnight. Previously frozen splenocytes were thawed and plated at 1×10⁵ to 2×10⁵ cells per well in media supplemented with 10% fetal bovine serum. Individual 15-mer peptides overlapping by 11 amino acids and representing the entire HA sequences from H5N1 influenza strains VN/1203/04 and A/HK/156/97 (Sigma Genosys, The Woodlands, Tex., USA) were dissolved in DMSO at 10 mg/ml and used in pools of 19-30 peptides (final concentration 3.33-5.26 μg/ml), pools of 9-10 peptides (5.0-5.5 μg/ml), or individually at 5.0 μg/ml as previously described. Brown, supra.

### 6.1.2. In Vivo Immunization In Mice

Four groups of seven BALB/c mice were immunized intramuscularly with 5×10¹⁰ viral particles of Ad.VN1203.HA, Ad.VN1203HA1, Ad.HK156HA1 (Ad.HAs), or empty vector Ad.ψ5, and received booster immunizations 14 days later. Initial screening of sera from immunized animals for antibodies by dot blot analysis performed on days 10, 24, and 31 identified HA-specific antibody responses in all immunized animals. Anti-influenza virus H5N1 neutralizing antibodies were then analyzed by a H5-specific ELISA or microneutralization assay. Rowe, T. et al. J. Clin. Microbial. 1999; 37(4):937-43. All groups vaccinated with an Ad.HA developed high titers of H5-specific IgG (Table 1). Neutralizing antibodies to VN/1203/04 or HK/156/97 were not detected in the groups immunized twice with control Ad.ψ5 or Ad.VN1203HA1. In contrast, neutralizing antibodies against the homologous subtype in mice immunized with Ad.VN1203.HA and some of the mice that received Ad.HK156HA1 were detected 10 days after the first dose, and titers were considerably increased 7 and 14 days after the boosting immunization. Notably, sera from mice immunized with Ad.VN1203.HA were able to cross-neutralize the HK/156/97 heterologous strain (Table 1).

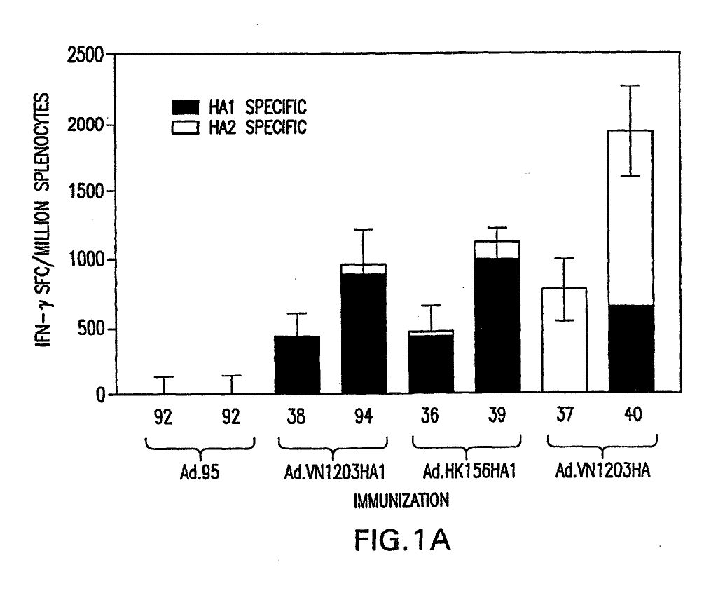

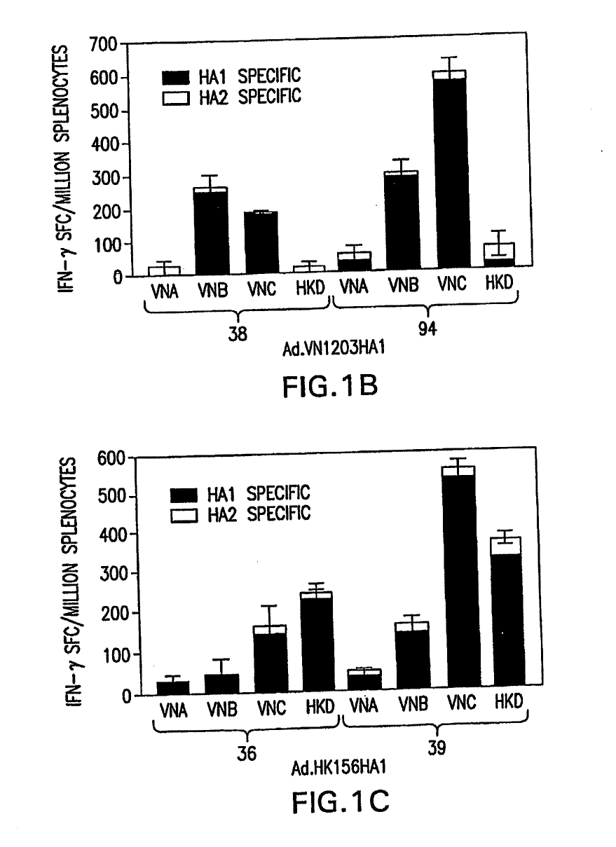

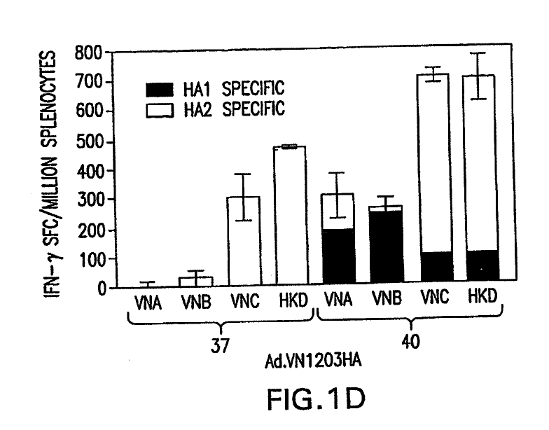

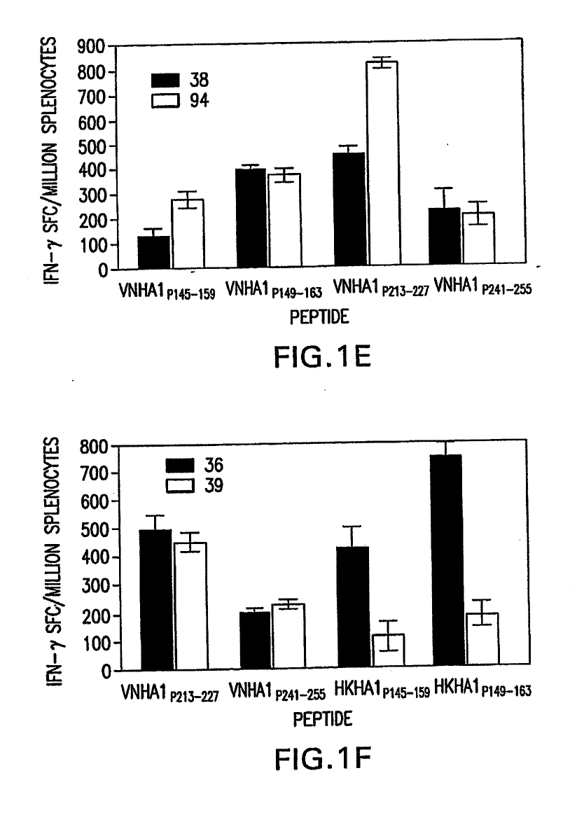

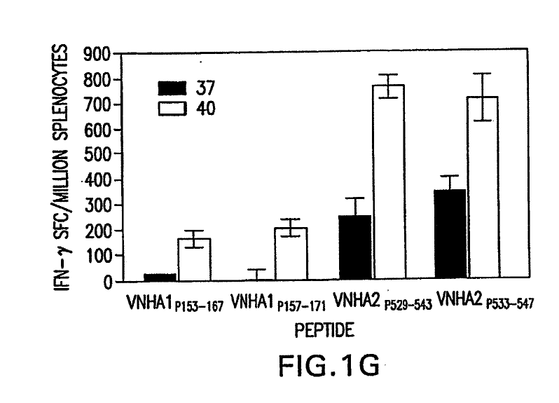

Vaccine-induced cellular immunity was measured through IFN-γ ELISPOT assays performed on two mice per group 9 days after receiving a third immunization. Overlapping 15mer peptides representing the entire VN/1203/04 HA protein and non-consensus sequences of HK/156/97 were pooled to evaluate the strength and breadth of immunity. Individual epitope-containing peptides were then identified through analysis of matrices in which each peptide was represented by two pools. Brown, K. et al. J Immunol. 2003; 171(12): 6875-82. All animals receiving intramuscular immunization against HA developed potent cellular responses reaching a peak intensity of 1 HA-specific T-cell per 500 freshly isolated splenocytes in mouse 40 (FIG. 1a). Cumulative cellular immune responses were generally HA region-specific, with only the Ad.VN1203.HA-immunized animals developing T-cell responses spanning the entire HA protein (FIG. 1a). Further analyses of strain-specific cellular immunity demonstrated that immunization with either Ad.VN1203HA1 or Ad.HK156HA1 was capable of inducing responses to the consensus A/VN/1203/04 sequences contained within pools VN1203-B and VN1203-C. In contrast, immunization with Ad.HK156HA1 was necessary to induce A/HK/156/97-specific pool HK156-D responses (FIGS. 1b, c). Detailed characterization of vaccine-induced immune responses identified four dominant peptide targets per immunization group (FIGS. 1e, f, g). Notably, responses against the immunodominant VN1203.HA1p₂₁₃₋₂₂₇ and subdominant VN1203.HA1P₂₄₁₋₂₅₅ regions were conserved regardless of HA1 immunization strain (FIGS. 1e, f). Ad.VN1203HA1 immunization-induced cellular immunity directed against the VN1203.HA1P₁₄₅₋₁₅₉/VN1203.HA1P₁₄₉₋₁₆₃ peptides suggested the presence of a shared epitope within this region. In addition, Ad.HK156HA1-immunized animals exhibited strain-specific immunity against the HK156.HA1p₁₄₅₋₁₅₉/HK156.HA1p₁₄₉₋₁₆₃ peptides unique to A/HK/156/97 (FIG. 1f). Interestingly, immunization with Ad.VN1203.HA encoding for the full-length HA protein altered the HA1-specific immune responses, potentially due to extra-epitopic modification or alternative peptide processing. Ad.VN1203.HA immunization revealed the presence of an immunodominant epitope in VN1203.HA2p₅₂₉₋₅₄₃/VN1203.HA2p₅₃₃₋₅₄₇ sequences contained within the HA2 portion of A/VN/1203/04 in addition to previously characterized responses towards the SFFRNVVWLIKK epitope contained within VN1203.HA1p₁₅₃₋₁₆₇ and VN1203.HA1p₁₅₇₋₁₇₁ (FIG. 1g), and in the non-consensus HK156.HA1p₁₅₃₋₁₆₇ peptide.

112 days after the second immunization, all mice were challenged by intranasal inoculation with 100 50% lethal infectious doses (LD₅₀) of VN/1203/04 virus. By three days post-challenge all animals immunized with control Ad.ψ5 vector experienced substantial weight loss, and subsequently died between days 6-9 post-challenge (Table 1). In contrast, animals inoculated with Ad.HAs showed no clinical signs of disease at 14 days post infection, and had only mild and transient loss of body weight.

The data demonstrate that replication-defective adenovirus-based vaccines may be effective as a first-line rapid response in the event of the emergence of a pandemic H5 strain.

### 6.3. Example 3

### In Vivo Immunization in Mice

### 6.3.1. Influenza Viruses

Influenza viruses used in this study were A/Hong Kong/156/97 (H5N1) (HK/156/97) and A/Vietnam/1203/2004 (H5N1) (VN/1203/04). Virus stocks were propagated at 37° C. in the allantoic cavity of 10-day-old embryonating hens' eggs for 26 hours and aliquoted and stored at negative 70° C. until use.

### 6.3.2. Gene Synthesis and Adenoviral Vectors Construction

HA, HA1 and HA2 genes from VN/1203/04 and HA1 gene from HK/156/97 were codon-optimized using the UpGene algorithm (www.vectorcore.pitt.edu/upgene.html) by overlapping oligonucleotides as previously described. Gao, supra. E1/E3-deleted adenoviral vectors expressing the codon-optimized genes were constructed using Cre-lox recombination into the adenoviral packaging cell line CRE8. Hardy, S. et al., J. Virol. 1997, 71:1842-1849. The recombinant adenoviruses were propagated in CRE8 cells, purified by cesium chloride density gradient centrifugation and dialysis, and stored at −70° C. Determination of adenovirus particle concentration was performed by spectrophotometer analysis using a validated assay based on Adenovirus Reference Material (ARM) obtained from the ATCC.

E1/E3-deleted adenovirus serotype 5-based vectors that express the codon-optimized 4 HA gene were generated as either the full length protein or the HA1 or HA2 subunits from the VN/1203/04 virus (Ad.VNHA, Ad.VNHA1, Ad.VNHA2). Additionally, a vector was generated containing the HA1 portion of the A/Hong Kong/156/1997 (H5N1) (HK/156/97) viral isolate (Ad.HKHA1). Generation of the recombinant adenoviral vectors was completed 36 days after acquiring the VN/1203/04 HA sequence, illustrating the rapid development and ease of manipulation necessary for adenoviral-based vaccine development.

### 6.3.3. Animal Experiments

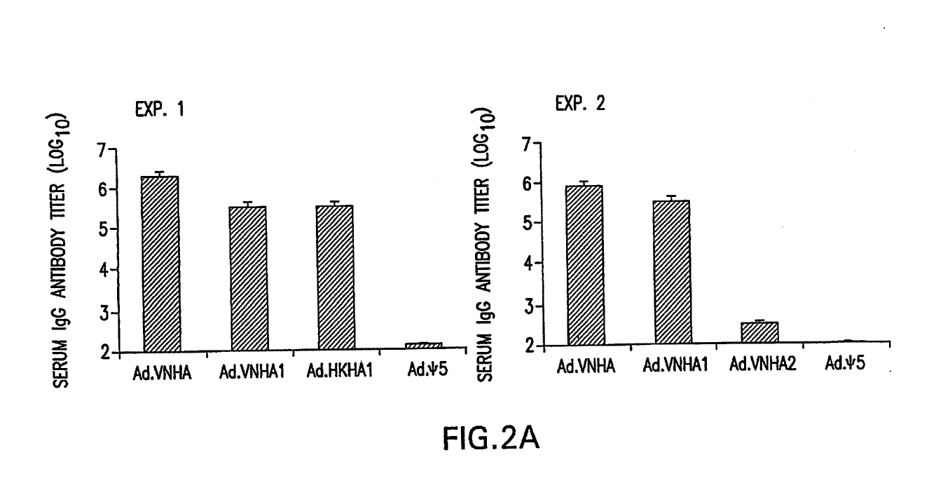

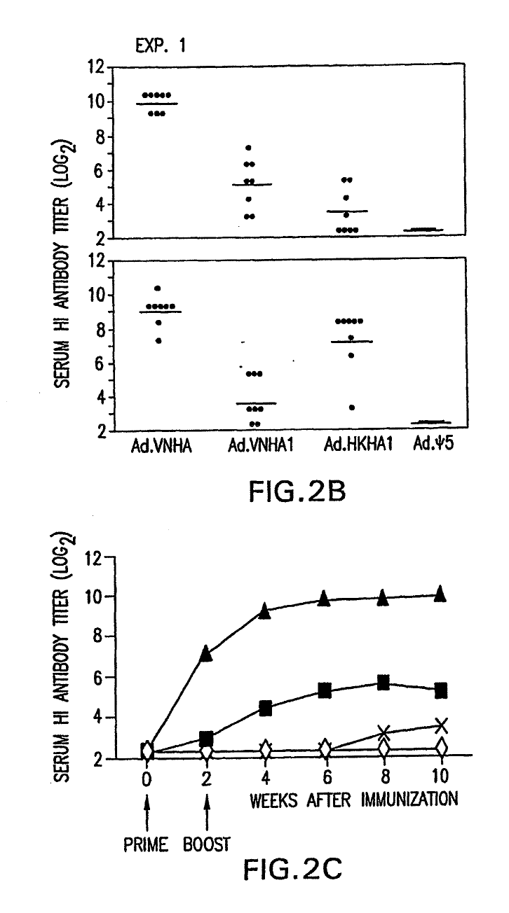

Six-week old BALB/c mice were used in murine experiments. Eight groups of 10 mice each were immunized with an intramuscular injection of 5×10¹⁰ virus particles of Ad.VNHA, Ad.VNHA1, Ad.HKHA1, Ad.VNHA2 and empty vector AdΨ5 at day 0 and day 14. Additional groups of mice were similarly vaccinated and boosted with Ad.VNHA, Ad.VNHA1, Ad.VNHA2, or empty vector Ad.Ψ5 (Exp. 2). All mice were bled to enable screening of sera for antibody responses, a surrogate marker of protection which can indicate immunogenicity. Karupiah, G. et al., Scand J Immunol. 1992, 36, 99-105. On week 10, eight weeks after the booster immunization, high titers of H5-specific antibodies were detected in all vaccinated animals except the Ad.VNHA2 group, which had titers more than three orders of magnitude lower than all other vaccinated groups (FIG. 2a).

The degree to which antibody responses could neutralize homologous VN/1203/04 and heterosubtypic HK/156/97 influenza strains was determined using the horse red blood cell hemagglutination inhibition (HI) assay. Stephenson et al., Virus Research 2004, 103, 91-95. Vaccination with full-length HA induced homologous and heterotypic antibody responses, whereas vaccination with Ad.VNHA1 or Ad.HKHA1 primarily induced antibody responses specific to the vaccinating strain (FIG. 2b). The modest antibody responses detected when HA1 was used as compared to the full-length protein is presumably because the HA1 subunit lacks trimeric conformation through the absence of HA2. The kinetics of serum HI responses suggest that a single immunization may be sufficient to achieve a high level anti-HA antibody responses (FIG. 2c).

At day 70 mice were lightly anesthetized with CO₂, and inoculated intranasally with 50 μl of 100 LD₅₀ of VN/1203/04 virus diluted in PBS. Mouse LD₅₀ titers were determined as previously described. Lu, X. H., et al., J. Virol. 1999, 73:5903-5911. To evaluate the degree of protection from challenge, eight vaccinated mice in each group were infected intranasally with 100 LD50 of VN/1203/04H5N1 virus. Five mice per group were observed daily for illness, weight loss and death for 14 days post infection, and three mice per group were sacrificed on day 3 or day 6 post infection for virus isolation, depending on the experiment.

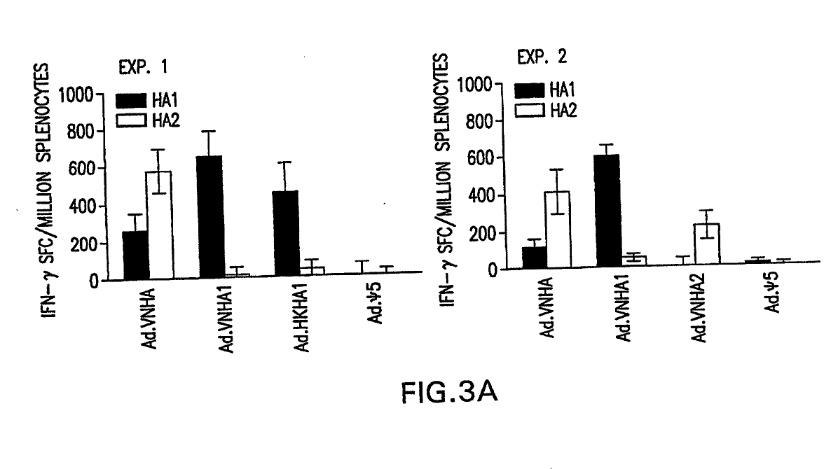

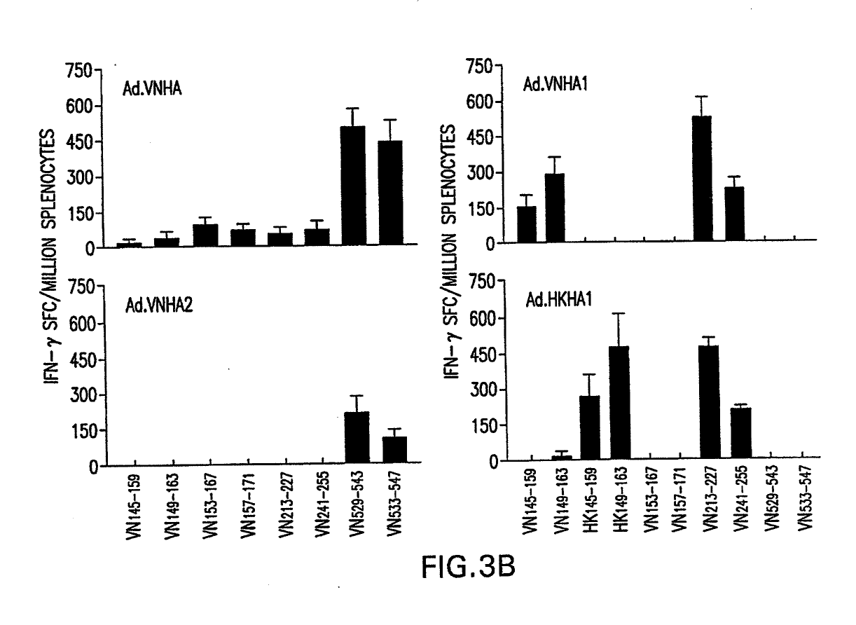

Given that vaccination induced variable degrees of humoral immunity, with the Ad.VNHA2-immunized group having a markedly reduced H5-specific antibody response, the cellular immune response to vaccination was next analyzed using the IFN-enzyme-linked immunospot (ELISPOT) assay in two mice per group after an additional boost immunization. Overlapping 15-mer peptides representing the entire VN/1203/04 HA protein and non-conserved sequences of HK/156/97 were pooled to evaluate the strength and breadth of immunity. Individual epitope-containing peptides were then identified through analysis of matrices in which each peptide was represented by two pools. Brown, K. et al., J Immunol. 2003; 171(12): 6875-82. All animals immunized with full-length HA or the HA1 or HA2 subunits developed strong cellular responses to HA peptides, reaching an average peak intensity of 1 HA-specific T-cell per 1,200 freshly isolated splenocytes in the Ad.VNHA group (FIG. 3A). Cumulative cellular immune responses were HA region-specific, with only the full length HA-immunized animals developing T-cell responses spanning both HA1 and HA2 (FIG. 3a). Detailed characterization of vaccine-induced immune responses identified both conserved and unique peptide targets (FIG. 3b). As expected, cellular responses against the conserved HA1 regions VN213-227 and VN241-255 were elicited regardless of HA1 immunization strain, whereas responses to peptides spanning amino acids 145-163, which differed between VN/1203/04 and HK/156/97, were limited to animals immunized with the respective subtype (FIG. 3b). Ad.VNHA2 immunization revealed the presence of an immunodominant epitope within HA2 represented by VN₅₂₉₋₅₄₃/VN₅₃₃₋₅₄₇ peptides. Immunization with Ad.VNHA induced a subdominant response to the previously identified SFFRNVVWLIKK epitope (Hioe, C. E. et al., J. Virol. 1990, 64, 6246-6251; Katz, J. M. et al., Biomed Pharmacother. 2000, 54, 178-187) contained within the HA1 peptides VN₁₅₃₋₁₆₇/VN₁₅₇₋₁₇₁. Immunization with Ad.VNHA altered the nature of HA1-specific immune responses seen when Ad.HA1 was the sole immunogen, generating more modest responses to VN₁₄₅₋₁₅₉/VN₁₄₉₋₁₆₃, VN₂₁₃₋₂₂₇ and VN₂₄₁₋₂₅₅ that were subdominant VN₅₂₉₋₅₄₃/VN₅₃₃₋₅₄₇ (FIG. 3b). These data demonstrate that adenovirus-based vaccination generates robust cellular immune responses to HA, which in the case of HA2 vaccination appears to be dominant to the humoral immune response.

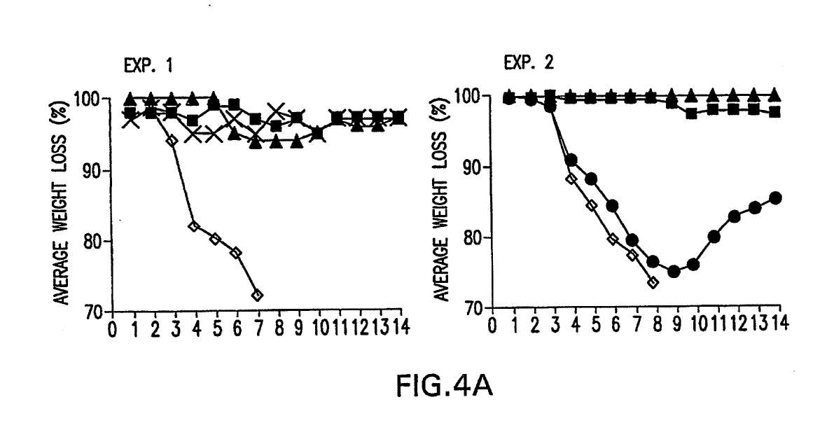

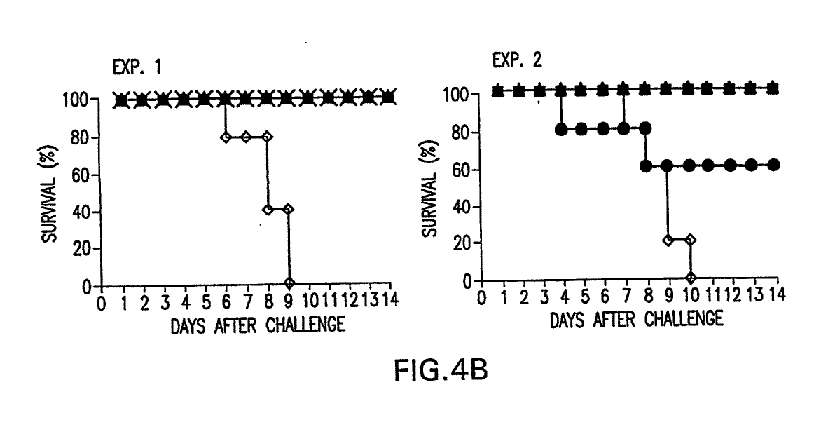

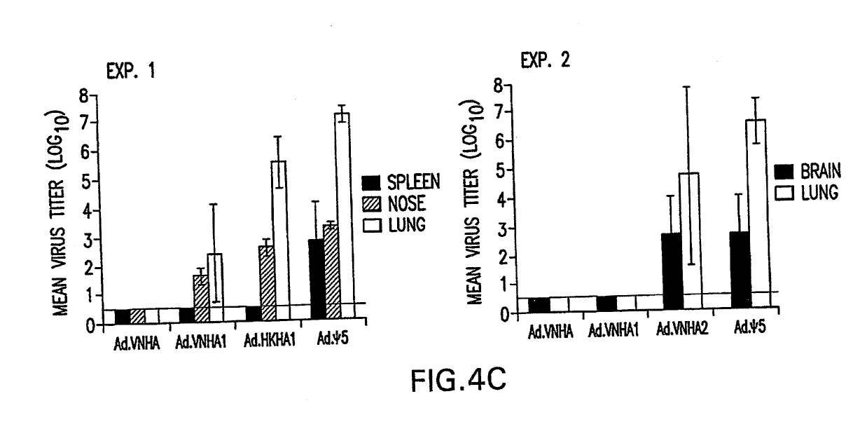

Eight weeks after the second immunization, all mice were challenged by intranasal inoculation with 100 50% lethal dose (LD₅₀) of VN/1203/04. All animals immunized with control Ad.Ψ5 vector experienced substantial weight loss beginning at day 3 post challenge and were dead by day 6-9 post challenge. In contrast, all animals immunized with Ad.VNHA, Ad.VNHA1 and Ad.HKHA1 showed only mild and transient loss of body weight and survived the lethal challenge (FIGS. 4a, b). All animals immunized with Ad.VNHA2 experienced substantial weight loss, but three out of five animals in this group regained weight after day 8 and fully recovered (FIGS. 4a, b). This recovery is notable given that vaccination with HA2 induced primarily cellular immune responses which previously have only been associated with enhanced viral clearance and recovery from influenza infection. Moss P., 2003, Dev Biol (Base1). 115, 31-37. At day 3 or 6 post challenge three animals per group were sacrificed for virus isolation. Infectious virus was isolated from multiple organs in the control vaccinated group and to various degrees in animals vaccinated with HA1 or HA2 subunits. In contrast, virus was isolated at extremely low levels on day 3 post infection (0.5 log₁₀ mean virus titer, Exp. 1) and not at all on day 6 post infection (<0.5 log₁₀ mean virus titer, Exp. 2) from organs from mice vaccinated with full-length HA (FIG. 4c).

### 6.5. Example 4

### In Vivo Immunization of Chickens

### 6.5.1. Methods

Influenza viruses used in this study were A/Hong Kong/156/97 (H5N1) (HK/156/97) and A/Vietnam/1203/2004 (H5N1) (VN/1203/04). Virus stocks were propagated as described in Example 3. Gene synthesis and adenoviral vector construction was performed as described in Example 3.

For avian studies, three-week old specific pathogen free single comb white leghorn chickens from an in house flock (SEPRL, USDA) were used. Groups of 10 chickens each were immunized with an intranasal or subcutaneous administration of 5×10¹⁰ virus particles of Ad.VNHA or AdΨ5. At 6 weeks of age chickens were challenged with 10⁶ EID₅₀ of VA/1203/04 virus intranasally through the choanal slit to determine protection. The chickens were observed daily for illness, weight loss and death for 14 days post infection. Serum was taken at 3, 6 and 8 weeks of age for detection of hemagglutination inhibition (HI) antibodies.

HI and ELISA assays. Immune sera from mice were collected by bleeding from the saphenous vein and were treated with receptor-destroying enzyme from Vibrio cholerae (Denka-Seiken, San Francisco, Calif., USA) before testing for the presence of H5-specific antibodies. Kendal, et al., In Concepts and procedures for laboratory-based influenza surveillance, Atlanta, CDC, B 17-35. (1982). The HI assay was performed using four HA units of virus and 1% horse red blood cells as described previously. Stephenson, supra. Influenza H5N1-specific IgG antibodies were detected by enzyme-linked immunosorbent assay (ELISA) as previously described (Katz, J. M., et al., J. Infect. Dis. 1997, 175:352-363) except that 1 μg/ml of purified baculovirus-expressed recombinant H5 HA protein from VN/1203/04 virus (Protein Sciences Corporation, Meriden, Conn., USA) was used to coat plates. ELISA end-point titers were expressed as the highest dilution that yielded an optical density greater than the 2 times the mean plus one standard deviation of similarly diluted negative control samples.

### 6.5.2. Results

Following the encouraging responses to vaccination and challenge in the murine model, the efficacy of adenovirus-based vaccination in chickens was evaluated, given the critical role this species plays in the spread of HPAI in southeast Asia. Chen, H. et al., Proc Natl Acad Sci USA. 2004, 101:10452-10457. The severity of H5N1 infection in chickens differs from mice as chickens rarely survive past the second day post challenge, whereas the median survival in naive mice is 8 days. The experiment was restricted to vaccination using full-length HA, given the superior protection noted in mice immunized with this vaccine.

As noted above, four groups of ten 3-week old chickens received one immunization subcutaneously or intranasally with 5×10¹⁰ viral particles of Ad.VNHA or empty vector Ad.Ψ5 and were challenged with an intranasal inoculation of 10⁶ EID₅₀ of VN/1203/04 21 days later. This virus dose is 10,000-fold higher than that given to mice and would likely represent a challenge significantly greater than chickens might experience in a natural outbreak. Vaccination induced HI antibodies to VN/1203/04 in all chickens in the subcutaneous immunization group which were boosted upon virus challenge (Table 2). All animals in this group survived challenge with no detectable clinical signs of disease (Table 2). In contrast, all control-immunized chickens died with a median survival of 1.8 days. Only one of the chickens immunized with Ad.VNHA intranasally had HI antibodies while, as a group, the chickens experienced 50% morbidity and 50% mortality following challenge (Table 2). The poorer protection afforded by intranasal immunization may reflect limited infection by adenovirus serotype 5 by this route as compared to the subcutaneous route. Oral and cloacal measurements of virus titers showed that subcutaneously administered vaccine greatly reduced replication of the challenge virus such that virus could not be detected in the gastrointestinal (GI) tract and levels were reduced by three orders of magnitude in the respiratory tract (Table 2).

It is widely accepted that novel influenza vaccination strategies are urgently needed, both to control spread of HPAI within fowl species and to prevent pandemic spread of HPAI in humans, should the capacity for human-to-human transmission emerge. The present invention demonstrates the ability of adenoviral-based immunization to induce both broad and potent HA-specific humoral and cellular immune responses which are able to confer protection against lethal intranasal challenge. Given the promise of adenoviral-based immunization in other vaccine applications (Shiver J. W. et al., Nature 2002, 415:331-335; Sullivan, N. J. et al., Nature 2003, 424:681-684) and promising results of an adenoviral-based immunization with an influenza HA vaccine in humans. A broadly cross-protective vaccination could be useful in domestic animals as well as humans, and adenoviral vectors may be a practical alternative to propagating vaccines using conventional methods in embryonated chicken eggs. Adam et al., J. Gen. Virol., 1995, 76(12):3153-3157.

These findings demonstrate the capacity of adenovirus-based immunization to induce broad and potent HA-specific humoral and cellular immune responses that provide protection from lethal intranasal challenge. Previous studies using inactivated whole H5N1 influenza virus vaccines in mice have indicated that strain-specific neutralizing antibodies provide long-lasting protection against homologous influenza virus challenge, (Subbarao, K. et al, Virology 2003, 305:192-200) but protection is limited against antigenically variant viral strains, such as heterotypic HA viral strains. The data presented herein suggest that adenoviral-based immunization stimulates both humoral and cellular responses that may offer broader protection covering antigenically drifted viral strains. Two recent studies have demonstrated the efficacy and immunogenicity of adenovirus-vectored influenza HA (H3N2) vaccines in swine and mice, and have revealed that cross-protection from heterotypic challenge can occur in the absence of neutralizing humoral immunity. Swayne et al., Avian Dis., 2003, 47:1047-1050; Wesley et al., Vaccine, 2004, 22:3427-3434.

Natural vector-specific immunity of some populations toward adenovirus serotype 5 (Nwanegbo, E. et al., Clin Diagn Lab Immunol. 2004, 11:351-357) could potentially reduce vaccine efficacy in the event that global vaccination against HPAI is implemented, adenovirus serotype 5-based vaccines against human immunodeficiency virus and Ebola virus have shown promise (Shiver J. W. et al., Nature 2002, 415:331-335; Sullivan, N. J. et al., Nature 2003, 424:681-684) and are being advanced to clinical trials. Importantly, vaccination was found to be highly effective in inducing anti-influenza neutralizing antibodies despite the presence of pre-existing anti-adenoviral antibodies, suggesting that vector-specific immunity may be overcome. Id. Alternatively, a wide range of different human and simian adenovirus serotypes are being developed as alternative vectors, which will likely negate the issue of pre-existing serotype 5-specific immunity. Farina et al., J. Virol., 2001, 75:11603-11613; Gao, W. et al., Gene Ther. 2003, 10: 1941-1949; Mei et al., J. Gen. Virol., 2003, 84:2061-2071; Pinto et al., J. Immunol., 2003, 171:6774-6779.

The present invention supports the development of replication-defective adenovirus-based vaccines as a first-line rapid response in the event of the pandemic spread of HPAI. Given the induction of protective immunity in chickens, widespread immunization of susceptible poultry would likely provide a significant barrier to the spread of HPAI and be economically advantageous. In addition, vaccination regimens could initially target high-risk human populations such as healthcare workers and animal handlers. Finally, in the worst case scenario of pandemic spread of lethal human disease, adenovirus-based immunizations could be utilized to complement traditional inactivated influenza vaccine technology, or by utilizing traditional vaccination strategies, such as in a ring vaccination strategy such as that implemented in the control of smallpox virus.

### REFERENCES

1. Abe, T., H. Takahashi, H. Hamazaki, N. Miyano-Kurosaki, Y. Matsuura, and H. Takaku. 2003. Baculovirus induces an innate immune response and confers protection from lethal influenza virus infection in mice. J Immunol 171:1133-1139.
2. Adam, M., W. Oualikene, H. Le Cocq, M. Guittet, and M. Eloit. 1995. Replication-defective adenovirus type 5 as an in vitro and in vivo gene transfer vector in chickens. J Gen Virol 76 (Pt 12):3153-3157.
3. Barouch, D. H., S. Santra, M. J. Kuroda, J. E. Schmitz, R. Plishka, A. Buckler-White, A. E. Gaitan, R. Zin, J. H. Nam, L. S. Wyatt, M. A. Lifton, C. E. Nickerson, B. Moss, D.C. Montefiori, V. M. Hirsch, and N. L. Letvin. 2001. Reduction of simian-human immunodeficiency virus 89.6P viremia in rhesus monkeys by recombinant modified vaccinia virus Ankara vaccination. J Virol 75:5151-5158.
4. Barouch, D. H., S. Santra, J. E. Schmitz, M. J. Kuroda, T. M. Fu, W. Wagner, M. Bilska, A. Craiu, X. X. Zheng, G. R. Krivulka, K. Beaudry, M. A. Lifton, C. E. Nickerson, W. L. Trigona, K. Punt, D.C. Freed, L. Guan, S. Dubey, D. Casimiro, A. Simon, M. E. Davies, M. Chastain, T. B. Strom, R. S. Gelman, D.C. Montefiori, M. G. Lewis, E. A. Emini, J. W. Shiver, and N. L. Letvin. 2000. Control of viremia and prevention of clinical AIDS in rhesus monkeys by cytokineaugmented DNA vaccination. Science 290:486-492.
5. Bender, B. S., C. A. Rowe, S. F. Taylor, L. S. Wyatt, B. Moss, and P. A. Small, Jr. 1996. Oral immunization with a replication-deficient recombinant vaccinia virus protects mice against influenza. J Virol 70:6418-6424.
6. Brown, K., W. Gao, S. Alber, A. Trichel, M. Murphey-Corb, S.C. Watkins, A. Gambotto, and S. M. Barratt-Boyes. 2003. Adenovirus-transduced dendritic cells injected into skin or lymph node prime potent simian immunodeficiency virus-specific T cell immunity in monkeys. J Immunol 171:6875-6882.
7. Check, E. 2005. Avian flu special: is this our best shot? Nature 435:404-406.
8. Chen, H., G. Deng, Z. Li, G. Tian, Y. Li, P. Jiao, L. Zhang, Z. Liu, R. G. Webster, and K. Yu. 2004. The evolution of H5N1 influenza viruses in ducks in southern China. Proc Natl Acad Sci USA 101:10452-10457.
9. Chen, J. D., Q. Yang, A. G. Yang, W. A. Marasco, and S. Y. Chen. 1996. Intraand extracellular immunization against HIV-1 infection with lymphocytes transduced with an AAV vector expressing a human anti-gp120 antibody. Hum Gene Ther 7:1515-1525.
10. Endo, A., S. Itamura, H. Iinuma, S. Funahashi, H. Shida, F. Koide, K. Nerome, and A. Oya. 1991. Homotypic and heterotypic protection against influenza virus infection in mice by recombinant vaccinia virus expressing the haemagglutinin or nucleoprotein of influenza virus. J Gen Virol 72 (Pt 3):699-703.
11. Epstein, S. L., W. P. Kong, J. A. Misplon, C. Y. Lo, T. M. Tumpey, L. Xu, and G. J. Nabel. 2005. Protection against multiple influenza A subtypes by vaccination with highly conserved nucleoprotein. Vaccine. 23(46-47):5404-10.
12. Farina, S. F., G. P. Gao, Z. Q. Xiang, J. J. Rux, R. M. Burnett, M. R. Alvira, J. Marsh, H. C. Ertl, and J. M. Wilson. 2001. Replication-defective vector based on a chimpanzee adenovirus. J Virol 75:11603-11613.
13. Fleeton, M. N., M. Chen, P. Berglund, G. Rhodes, S. E. Parker, M. Murphy, G. J. Atkins, and P. Liljestrom. 2001. Self-replicative RNA vaccines elicit protection against influenza A virus, respiratory syncytial virus, and a tickborne encephalitis virus. J Infect Dis 183:1395-1398.
14. Gao, W., P. D. Robbins, and A. Gambotto. 2003. Human adenovirus type 35: nucleotide sequence and vector development. Gene Ther 10: 1941-1949.
15. Gao, W., A. Rzewski, H. Sun, P. D. Robbins, and A. Gambotto. 2004. UpGene: Application of a web-based DNA codon optimization algorithm. Biotechnol Prog 20:443-448.
16. Hardy, S., M. Kitamura, T. Harris-Stansil, Y. Dai, and M. L. Phipps. 1997. Construction of adenovirus vectors through Cre-lox recombination. J Virol 71:1842-1849.
17. Hioe, C. E., N. Dybdahl-Sissoko, M. Philpott, and V. S. Hinshaw. 1990. Overlapping cytotoxic T-lymphocyte and B-cell antigenic sites on the influenza virus H5 hemagglutinin. J Virol 64:6246-6251.
18. Karupiah, G., A. J. Ramsay, I. A. Ramshaw, and R. V. Blanden. 1992. Recombinant vaccine vector-induced protection of athymic, nude mice from influenza A virus infection. Analysis of protective mechanisms. Scand J Immunol 36:99-105.
19. Katz, J. M., X. Lu, A. M. Frace, T. Morken, S. R. Zaki, and T. M. Tumpey. 2000. Pathogenesis of and immunity to avian influenza A H5 viruses. Biomed Pharmacother 54:178-187.
20. Katz, J. M., X. Lu, S. A. Young, and J. C. Galphin. 1997. Adjuvant activity of the heat-labile enterotoxin from enterotoxigenic Escherichia coli for oral administration of inactivated influenza virus vaccine. J Infect Dis 175:352-363.
21. Kendal, A. P., Skehel, J. J., Pereira, M. S., In Concepts and procedures for laboratory-based influenza surveillance, Atlanta, CDC, B17-35. (1982).
22. Kodihalli, S., D. L. Kobasa, and R. G. Webster. 2000. Strategies for inducing protection against avian influenza A virus subtypes with DNA vaccines. Vaccine 18:2592-2599.
23. Li, K. S., Y. Guan, J. Wang, G. J. Smith, K. M. Xu, L. Duan, A. P. Rahardjo, P. Puthavathana, C. Buranathai, T. D. Nguyen, A. T. Estoepangestie, A. Chaisingh, P. Auewarakul, H. T. Long, N. T. Hanh, R. J. Webby, L. L. Poon, H. Chen, K. F. Shortridge, K. Y. Yuen, R. G. Webster, and J. S. Peiris. 2004. Genesis of a highly pathogenic and potentially pandemic H5N1 influenza virus in eastern Asia. Nature 430:209-213.
24. Lipatov, A. S., R. J. Webby, E. A. Govorkova, S. Krauss, and R. G. Webster. 2005. Efficacy of H5 influenza vaccines produced by reverse genetics in a lethal mouse model. Infect Dis 191:1216-1220.
25. Lu, X., T. M. Tumpey, T. Morken, S. R. Zaki, N. J. Cox, and J. M. Katz. 1999. A mouse model for the evaluation of pathogenesis and immunity to influenza A (H5N1) viruses isolated from humans. J Virol 73:5903-5911.
26. Mei, Y. F., J. Skog, K. Lindman, and G. Wadell. 2003. Comparative analysis of the genome organization of human adenovirus 11, a member of the human adenovirus species B, and the commonly used human adenovirus 5 vector, a member of species C. J Gen Virol 84:2061-2071.
27. Moss, P. 2003. Cellular immune responses to influenza. Dev Biol (Base1) 115:31-37.
28. Nicholson, K. G., J. M. Wood, and M. Zambon. 2003. Influenza. Lancet 362:1733-1745.
29. Nwanegbo, E., E. Vardas, W. Gao, H. Whittle, H. Sun, D. Rowe, P. D. Robbins, and A. Gambotto. 2004. Prevalence of neutralizing antibodies to adenoviral serotypes 5 and 35 in the adult populations of The Gambia, South Africa, and the United States. Clin Diagn Lab Immunol 11:351-357.
30. Pinto, A. R., J. C. Fitzgerald, W. Giles-Davis, G. P. Gao, J. M. Wilson, and H. C. Ertl. 2003. Induction of CD8+ T cells to an HIV-1 antigen through a prime boost regimen with heterologous E1-deleted adenoviral vaccine carriers. J Immunol 171:6774-6779.
31. Rowe, T. et al. Detection of antibody to avian influenza A (H5N1) virus in human serum by using a combination of serologic assays. J. Clin. Microbial. 1999; 37(4): 937-43.
32. Shiver, J. W., and E. A. Emini. 2004. Recent advances in the development of HIV-1 vaccines using replication-incompetent adenovirus vectors. Annu Rev Med 55:355-372.
33. Shiver, J. W., T. M. Fu, L. Chen, D. R. Casimiro, M. E. Davies, R. K. Evans, Z. Q. Zhang, A. J. Simon, W. L. Trigona, S. A. Dubey, L. Huang, V. A. Harris, R. S. Long, X. Liang, L. Handt, W. A. Schleif, L. Zhu, D.C. Freed, N. V. Persaud, L. Guan, K. S. Punt, A. Tang, M. Chen, K. A. Wilson, K. B. Collins, G. J. Heidecker, V. R. Fernandez, H. C. Perry, J. G. Joyce, K. M. Grimm, J. C. Cook, P. M. Keller, D. S. Kresock, H. Mach, R. D. Troutman, L. A. Isopi, D. M. Williams, Z. Xu, K. E. Bohannon, D. B. Volkin, D.C. Montefiori, A. Miura, G. R. Krivulka, M. A. Lifton, M. J. Kuroda, J. E. Schmitz, N. L. Letvin, M. J. Caulfield, A. J. Bett, R. Youil, D.C. Kaslow, and E. A. Emini. 2002. Replication-incompetent adenoviral vaccine vector elicits effective antiimmunodeficiency-virus immunity. Nature 415:331-335.
34. Stephenson, I., J. M. Wood, K. G. Nicholson, A. Charlett, and M. C. Zambon. 2004. Detection of anti-H5 responses in human sera by HI using horse erythrocytes following MF59-adjuvanted influenza A/Duck/Singapore/97 vaccine. Virus Res 103:91-95.
35. Sullivan, N. J. et al. Accelerated vaccination for Ebola virus haemorrhagic fever in non-human primates. Nature. 424, 681-684 (2003).
36. Subbarao, K., H. Chen, D. Swayne, L. Mingay, E. Fodor, G. Brownlee, X. Xu, X. Lu, J. Katz, N. Cox, and Y. Matsuoka. 2003. Evaluation of a genetically modified reassortant H5N1 influenza A virus vaccine candidate generated by plasmid-based reverse genetics. Virology 305:192-200.
37. Sullivan, N. J., A. Sanchez, P. E. Rollin, Z. Y. Yang, and G. J. Nabel. 2000. Development of a preventive vaccine for Ebola virus infection in primates. Nature 408:605-609.
38. Swayne, D. E., M. Garcia, J. R. Beck, N. Kinney, and D. L. Suarez. 2000. Protection against diverse highly pathogenic H5 avian influenza viruses in chickens immunized with a recombinant fowlpox vaccine containing an H5 avian influenza hemagglutinin gene insert. Vaccine 18:1088-1095.
39. Swayne, D. E., D. L. Suarez, S. Schultz-Cherry, T. M. Tumpey, D. J. King, T. Nakaya, P. Palese, and A. Garcia-Sastre. 2003. Recombinant paramyxovirus type 1-avian influenza-H7 virus as a vaccine for protection of chickens against influenza and Newcastle disease. Avian Dis 47:1047-1050.
40. Van Kampen, K. R., Z. Shi, P. Gao, J. Zhang, K. W. Foster, D. T. Chen, D. Marks, C. A. Elmets, and D.C. Tang. 2005. Safety and immunogenicity of adenovirus-vectored nasal and epicutaneous influenza vaccines in humans. Vaccine 23:1029-1036.
41. Webby, R. J., D. R. Perez, J. S. Coleman, Y. Guan, J. H. Knight, E. A. Govorkova, L. R. McClain-Moss, J. S. Peiris, J. E. Rehg, E. I. Tuomanen, and R. G. Webster. 2004. Responsiveness to a pandemic alert: use of reverse genetics for rapid development of influenza vaccines. Lancet 363:1099-1103.
42. Wesley, R. D., M. Tang, and K. M. Lager. 2004. Protection of weaned pigs by vaccination with human adenovirus 5 recombinant viruses expressing the hemagglutinin and the nucleoprotein of H3N2 swine influenza virus. Vaccine 22:3427-3434.
43. Yuen, K. Y., P. K. Chan, M. Peiris, D. N. Tsang, T. L. Que, K. F. Shortridge, P. T. Cheung, W. K. To, E. T. Ho, R. Sung, and A. F. Cheng. 1998. Clinical features and rapid viral diagnosis of human disease associated with avian influenza A H5N1 virus. Lancet 351:467-471.

Various references are cited herein, which are hereby incorporated by reference in their entireties

TABLE 1
Protective efficacy of Ad.HAs vaccines against
a lethal infection with VN/1203/04 virus

| | | IgG | Neutralizing Antibody | | |
|---|---|---|---|---|---|
| Vaccine | Mouse | HK/156 | Titers^{a} | | Death (D) or |
| Group | N° | rHA^{a} | HK/156 | VN/1203 | Survival (S)^{b} |
| Ad.VNl203 | 41 | 6.9 | 1280 | 640 | S |
| HA | 42 | 7.9 | 640 | 320 | S |
| | 43 | 6.0 | 640 | 640 | S |
| | 44 | 6.0 | 640 | 640 | S |
| | 45 | 5.8 | 1280 | 640 | S |
| Ad.HK156 | 76 | 7.9 | <40 | <40 | S |
| HA1 | 77 | 7.6 | <40 | <40 | S |
| | 78 | 6.9 | 1280 | <40 | S |
| | 79 | 6.6 | <40 | <40 | S |
| | 80 | 6.4 | 160 | <40 | S |
| Ad.VNl203 | 81 | 7.5 | <40 | <40 | S |
| HA1 | 82 | 6.5 | <40 | <40 | S |
| | 83 | 4.9 | <40 | <40 | S |
| | 84 | 5.4 | <40 | <40 | S |
| | 85 | 5.5 | <40 | <40 | S |
| Ad.ψ5 | 86 | 2.0 | <40 | <40 | D |
| | 87 | 2.8 | <40 | <40 | D |
| | 88 | 2.0 | <40 | <40 | D |
| | 89 | 2.0 | <40 | <40 | D |
| | 90 | 2.0 | <40 | <40 | D |

^{a}Sera collected 4 weeks after immunization were treated with RDE and tested for the presence of IgG antibody by ELISA using rHA from HK/156/97 virus or neutralizing antibody using infectious HK/156/97 or VN/1203/04 virus.
^{b}Mice were challenged intranasally with 100 LD₅₀ of VN/1203/04 virus and monitored for 14 days. Control mice (86-90) died on days 6-9 after challenge.

TABLE 2
Efficacy of VN/1203/04 vaccination in chickens

| | | | | Serum HI antibody titer (GMT) | | |
|---|---|---|---|---|---|---|
| Group | Route | Morbidity | Mortality (MDT) | d 0 PV | d 21 PV | d 14 PC |
| Ad.Y5 | IN | 10/10 | 10/10 (1.8) | 0/10 | 0/10 | NA |
| Ad.Y5 | SQ | 10/10 | 10/10 (1.8) | 0/10 | 0/10 | NA |
| Ad.VNHA | IN | 5/10 | 5/10 (6.0) | 0/10 | 1/10 (4) | 5/5 (97) |
| Ad.VNHA | SQ | 0/10 | 0/10 | 0/10 | 10/10 (13) | 10/10 (315) |

Data are shown as ratio of number of animals affected to total number of animals per group. GMT, geometric mean reciprocal endpoint titer; MDT, median time to death in days; PV, post vaccination; PC, post challenge; IN, intranasal; SQ, subcutaneous; NA, not available.

We claim:

1. A replication-defective adenoviral vector comprising a nucleic acid encoding an influenza A polypeptide derived from A/Vietnam/1203/2004 (H5N1) operably linked to expression control sequences such that the influenza A polypeptide can be expressed, wherein upon introduction of the vector into a subject, the expressed polypeptide induces the subject to produce antibodies that bind to influenza.

2. The replication-defective adenoviral vector of claim 1, wherein the influenza A polypeptide is hemagglutinin (HA).

3. The replication-defective adenoviral vector of claim 2, wherein the influenza A polypeptide is hemagglutinin subunit 1 (HA1).

4. The replication-defective adenoviral vector of claim 1, wherein the subject is a mammal.

5. The replication-defective adenoviral vector of claim 4, wherein the subject is a human.

6. The replication-defective adenoviral vector of claim 1, wherein the subject is a bird.

7. The replication-defective adenoviral vector of claim 6, wherein the subject is a chicken.

8. The replication-defective adenoviral vector of claim 1, wherein the adenoviral vector is deficient in E1 or E3.

9. A vaccine composition comprising: (1) a replication-defective adenoviral vector comprising a nucleic acid encoding an influenza A polypeptide derived from A/Vietnam/1203/2004 (H5N1) operably linked to expression control sequences such that the influenza A polypeptide can be expressed, wherein upon introduction of the vector into a subject, the expressed polypeptide induces the subject to produce antibodies that bind to influenza; and (2) a pharmaceutically acceptable carrier.

10. The vaccine of claim 9, wherein the influenza A polypeptide is hemagglutinin (HA).

11. The vaccine of claim 10, wherein the influenza A-polypeptide is hemagglutinin subunit 1 (HA1).

12. The vaccine of claim 9, wherein the subject is a mammal.

13. The vaccine of claim 12, wherein the subject is a human.

14. The vaccine of claim 9, wherein the subject is a bird.

15. The vaccine of claim 14, wherein the subject is a chicken.
